(19) United States
(12) Patent Application Publication
(10) Pub. No.: US 2008/0090809 A1
(43) Pub. Date: Apr. 17, 2008

(54) PYRAZOLE COMPOUNDS

(75) Inventors: Kak-Shan Shia, Taipei (TW); Chia-Liang Tai, Hsinchu (TW); Jing-po Tsao, Hsinchu (TW); Wan-ping Hsieh, Hsinchu (TW); Shi-liang Tseng, Hsinchu (TW); Yu-Sheng Chao, Warren, NJ (US); Ming-Shiu Hung, Luchu (TW)

Correspondence Address: OCCHIUTI ROHLICEK & TSAO, LLP, 10 FAWCETT STREET, CAMBRIDGE, MA, 02138, US

(73) Assignee: National Health Research Institutes, Zhunan Town (TW)

(21) Appl. No.: 11/866,233

(22) Filed: Oct. 2, 2007

Related U.S. Application Data
(60) Provisional application No. 60/848,742, filed on Oct. 2, 2006.

(51) Int. Cl. A61K 31/55; A61K 31/415; A61P 29/00; C07D 231/10; C07D 403/14; C07D 401/14; A61P 35/00; A61K 31/4453

(52) U.S. Cl. 514/217.09; 514/406; 540/603; 546/211; 548/365.7; 548/374.1

(57) ABSTRACT

This invention relates to pyrazole compounds of formula (I) shown below: 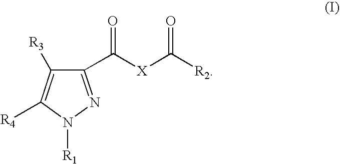 Each variable in formula (I) is defined in the specification. These compounds can be used to treat cannabinoid-receptor mediated disorders.

### CROSS-REFERENCE TO RELATED APPLICATIONS

This application claims the benefit of U.S. Provisional Application No. 60/848,742, filed Oct. 2, 2006. The contents of the foregoing application are hereby incorporated by reference in its entirety.

### BACKGROUND

Cannabinoids isolated from Cannabis sativa have been recognized for centuries as therapeutic agents. For example, they have been utilized in treating analgesia, muscle relaxation, appetite stimulation, and anti-convulsion. Recent studies also indicate their potential therapeutic effects in treating cancer and alleviating the symptoms of chronic inflammatory diseases, such as rheumatism and multiple sclerosis.

The actions of cannabinoids are mediated by at least two types of the cannabinoid receptors, CB1 and CB2 receptors, both of which belong to the G-protein-coupled receptor (GPCR) superfamily. CB1 receptor is predominantly expressed in brain to mediate inhibition of transmitter release and CB2 receptor is primarily expressed in immune cells to modulate immune response. See Matsuda et al., Nature (1990) 346:561 and Munro et al., Nature (1993) 365:61.

Compared to other GPCRs, CB1 receptor is typically expressed at higher levels. In the central nervous system, it is highly expressed halo or C₁-C₁₀ alkyl in cerebral cortex, hippocampus, basal ganglia, and cerebellum, but has relatively low levels in hypothalamus and spinal cord. See, e.g., Howlett et al., Pharmacol Rev (2002) 54:161. Its functions affect many neurological and psychological phenomena, such as mood, appetite, emesis control, memory, spatial coordination muscle tone, and analgesia. See, e.g., Goutopoulos et al., Pharmacol Ther (2002) 95:103. Other than the central nervous system, it is also present in several peripheral organs, such as gut, heart, lung, uterus, ovary, testis, and tonsils. See, e.g., Galiègue et al., Eur J Biochem (1995) 232:54.

CB2 receptor is 44% identical to CB1 receptor with a 68% identity in the trans-membrane regions. See Munro et al., Nature (1993) 365:61. Compared to CB1 receptor, CB2 receptor has a more limited distribution with high expression in spleen and tonsils, and low expression in lung, uterus, pancreas, bone marrow, and thymus. Among immune cells, B cells express CB2 receptor at the highest level, followed in order by natural killer cells, monocytes, polymorphonuclear neutrophils, and T lymphocytes. See Galiègue et al., Eur J Biochem (1995) 232:54. Activation of CB2 receptor has been shown to have analgesic effects in inflammatory models involved in neurodegeneration diseases (such as Alzheimer's disease), and play a role in the maintenance of bone density and progression of atherosclerotic lesions. See, e.g., Malan et al., Pain (2001) 93:239; Benito et al., J Neurosci (2003) 23:11136; Ibrahim et al., Proc Natl Acad Sci USA (2003) 100:10529; Idris et al., Nat Med (2005) 11:774; and Steffens et al., Nature (2005) 434:782.

### SUMMARY

This invention is based on the discovery that certain pyrazole compounds are effective in treating cannabinoid-receptor mediated disorders.

In one aspect, this invention features pyrazole compounds of formula (I):

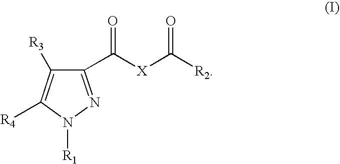

In this formula, X is C(RₐR_{b}) or N(Rₐ), in which each of Rₐ and R_{b}, independently, is H, C₁-C₁₀ alkyl, C₃-C₂₀ cycloalkyl, C₁-C₂₀ heterocycloalkyl, aryl, or heteroaryl; R₂ is H, halo, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, or NR_{c}R_{d}, in which each of R_{c}, and R_{d}, independently, is H, C₁-C₁₀ alkyl, C₃-C₂₀ cycloalkyl, C₁-C₂₀ heterocycloalkyl, aryl, or heteroaryl; and each of R₁, R₃, and R₄, independently, is H, halo, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

Referring to formula (I), a subset of the pyrazole compounds described above are those in which X can be CH₂ or NH, R₁ can be aryl substituted with halo (e.g., 2,4-dichlorophenyl), R₄ can be aryl or heteroaryl, R₂ can be C₁-C₁₀ alkyl, C₃-C₂₀ cycloalkyl, C₁-C₂₀ heterocycloalkyl, aryl, or NR_{c}R_{d}, in which each of R^{c} and R_{d}, independently, is H, C₁-C₁₀ alkyl, C₃-C₂₀ cycloalkyl, C₁-C₂₀ heterocycloalkyl, aryl, or heteroaryl, and R₃ can be H, halo, or C₁-C₁₀ alkyl.

The term “alkyl” refers to a saturated, linear or branched hydrocarbon moiety, such as —CH₃ or —CH(CH₃)₂. The term “alkenyl” refers to a linear or branched hydrocarbon moiety that contains at least one double bond, such as —CH═CH—CH₃. The term “alkynyl” refers to a linear or branched hydrocarbon moiety that contains at least one triple bond, such as —C≡C—CH₃. The term “cycloalkyl” refers to a saturated, cyclic hydrocarbon moiety, such as cyclohexyl. The term “cycloalkenyl” refers to a non-aromatic, cyclic hydrocarbon moiety that contains at least one double bond, such as cyclohexenyl. The term “heterocycloalkyl” refers to a saturated, cyclic moiety having at least one ring heteroatom (e.g., N, O, or S), such as 4-tetrahydropyranyl. The term “heterocycloalkenyl” refers to a non-aromatic, cyclic moiety having at least one ring heteroatom (e.g., N, O, or S) and at least one ring double bond, such as pyranyl. The term “aryl” refers to a hydrocarbon moiety having one or more aromatic rings. Examples of aryl moieties include phenyl (Ph), phenylene, naphthyl, naphthylene, pyrenyl, anthryl, and phenanthryl. The term “heteroaryl” refers to a moiety having one or more aromatic rings that contain at least one heteroatom (e.g., N, O, or S). Examples of heteroaryl moieties include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl.

Alkyl, alkenyl, alkynyl, cycloalkyl, cycloalkenyl, heterocycloalkyl, heterocycloalkenyl, aryl, and heteroaryl mentioned herein include both substituted and unsubstituted moieties, unless specified otherwise. Possible substituents on cycloalkyl, cycloalkenyl, heterocycloalkyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, nitroso, azido, acyl, thioacyl, acyloxy, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

In still another aspect, this invention features a method for treating a cannabinoid-receptor mediated disorder. The method includes administering to a subject in need thereof an effective amount of one or more pyrazole compounds of formula (I) shown above. Examples of cannabinoid-receptor mediated disorders include liver fibrosis, hair loss, obesity, metabolic syndrome (e.g., syndrome X), hyperlipidemia, type II diabetes, atherosclerosis, substance addiction (e.g., alcohol addiction or nicotine addiction), depression, motivational deficiency syndrome, learning or memory dysfunction, analgesia, haemorrhagic shock, ischemia, liver cirrhosis, neuropathic pain, antiemesis, high intraocular pressure, bronchodilation, osteoporosis, cancer (e.g., prostate cancer, lung cancer, breast cancer, or head and neck cancer), a neurodegenerative disease (e.g., Alzheimer's disease or Parkinson's disease), or an inflammatory disease.

The term “treating” or “treatment” refers to administering one or more pyrazole compounds to a subject, who has an above-described disorder, a symptom of such a disorder, or a predisposition toward such a disorder, with the purpose to confer a therapeutic effect, e.g., to cure, relieve, alter, affect, ameliorate, or prevent the above-described disorder, the symptom of it, or the predisposition toward it.

In addition, this invention encompasses a pharmaceutical composition that contains an effective amount of at least one of the above-mentioned pyrazole compounds and a pharmaceutically acceptable carrier.

The pyrazole compounds described above include the compounds themselves, as well as their salts, prodrugs, and solvates, if applicable. A salt, for example, can be formed between an anion and a positively charged group (e.g., amino) on a pyrazole compound. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a pyrazole compound. Suitable cations include sodium ion, potassium ion, magnesium ion, calcium ion, and an ammonium cation such as tetramethylammonium ion. The pyrazole compounds also include those salts containing quaternary nitrogen atoms. Examples of prodrugs include esters and other pharmaceutically acceptable derivatives, which, upon administration to a subject, are capable of providing active pyrazole compounds. A solvate refers to a complex formed between an active pyrazole compound and a pharmaceutically acceptable solvent. Examples of pharmaceutically acceptable solvents include water, ethanol, isopropanol, ethyl acetate, acetic acid, and ethanolamine.

Also within the scope of this invention is a composition containing one or more of the pyrazole compounds described above for use in treating an above-described disorder, and the use of such a composition for the manufacture of a medicament for the just-mentioned treatment.

The details of one or more embodiments of the invention are set forth in the description below. Other features, objects, and advantages of the invention will be apparent from the description and from the claims.

### DETAILED DESCRIPTION

The pyrazole compounds described above can be prepared by methods well known in the art, such as methods similar to those described in U.S. Provisional Application Ser. No. 60/819,147. A synthesized pyrazole compound can be purified by a suitable method such as column chromatography, high-pressure liquid chromatography, or recrystallization.

The pyrazole compounds mentioned herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

Also within the scope of this invention is a pharmaceutical composition containing an effective amount of at least one pyrazole compound described above and a pharmaceutical acceptable carrier. Further, this invention covers a method of administering an effective amount of one or more of the pyrazole compounds to a patient having a disease described in the summary section above. “An effective amount” refers to the amount of an active pyrazole compound that is required to confer a therapeutic effect on the treated subject. Effective doses will vary, as recognized by those skilled in the art, depending on the types of diseases treated, route of administration, excipient usage, and the possibility of co-usage with other therapeutic treatment.

To practice the method of the present invention, a composition having one or more pyrazole compounds can be administered parenterally, orally, nasally, rectally, topically, or buccally. The term “parenteral” as used herein refers to subcutaneous, intracutaneous, intravenous, intrmuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more active pyrazole compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active pyrazole compound. Examples of other carriers include colloidal silicon oxide, magnesium stearate, cellulose, sodium lauryl sulfate, and D&C Yellow # 10.

The pyrazole compounds described above can be preliminarily screened for their efficacy in treating above-described diseases by an in vitro assay and then confirmed by animal experiments and clinic trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

Below are exemplary compounds of the invention, which are grouped into four classes. 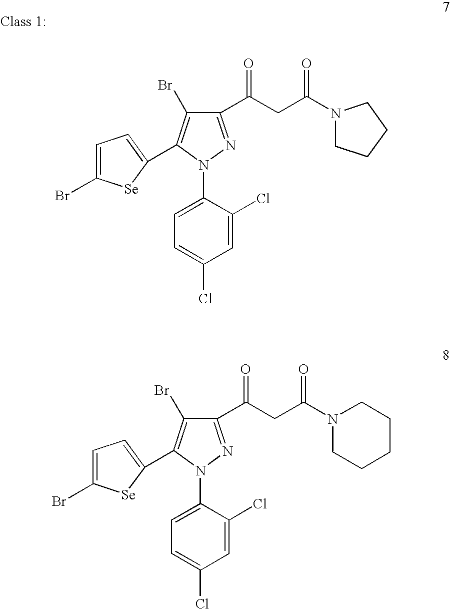 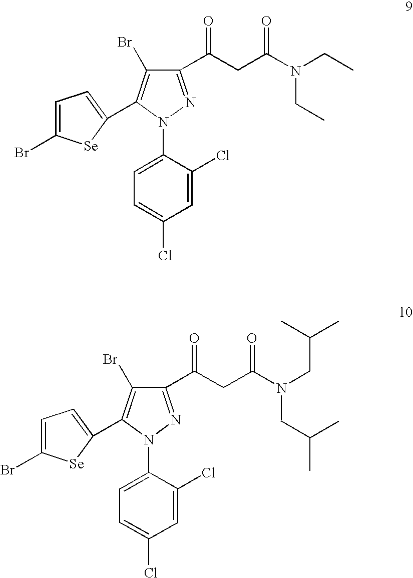 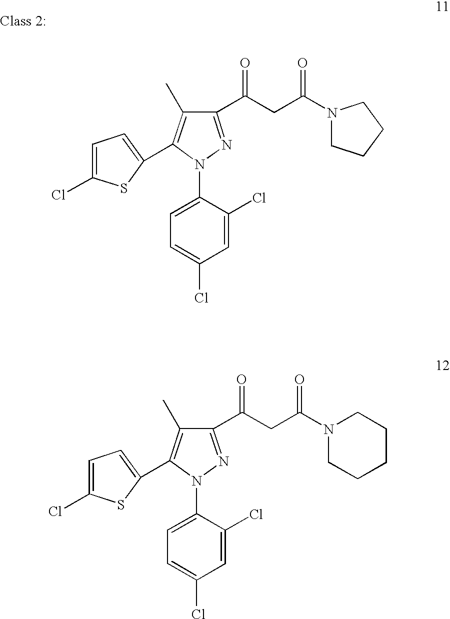 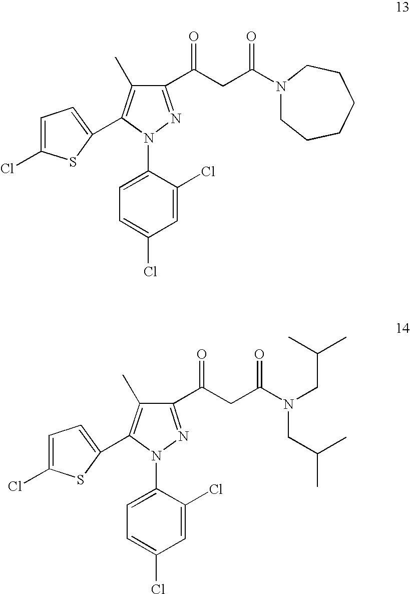 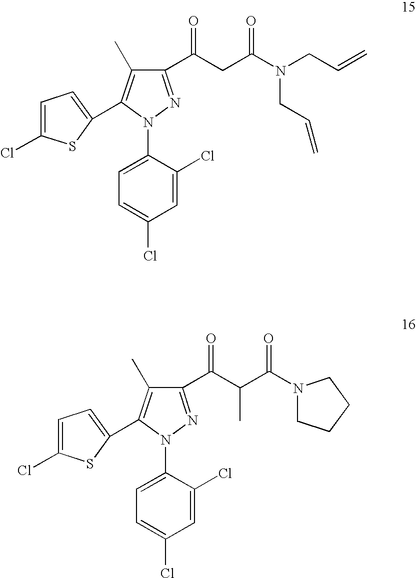 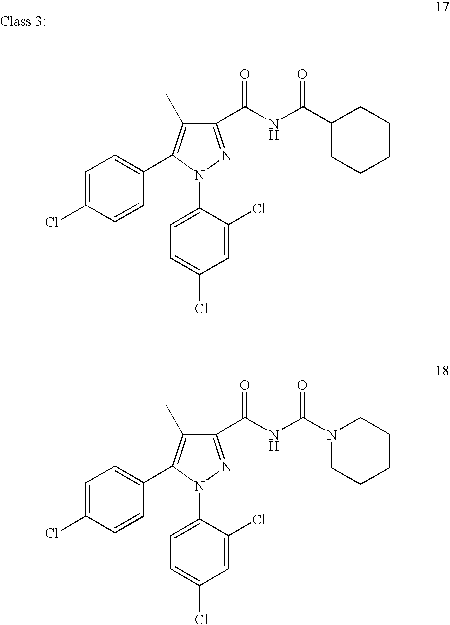 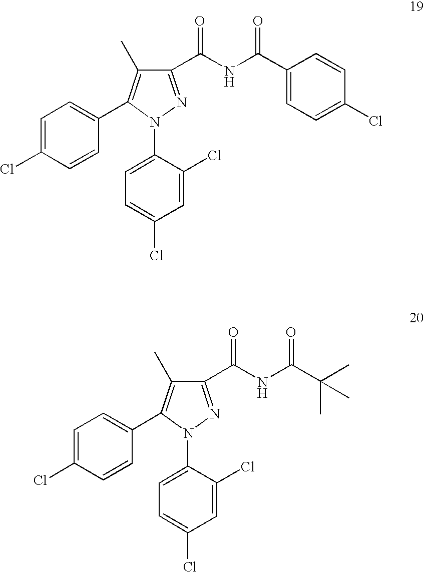 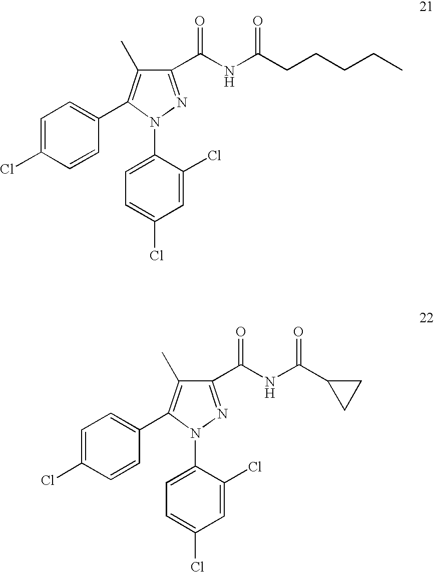 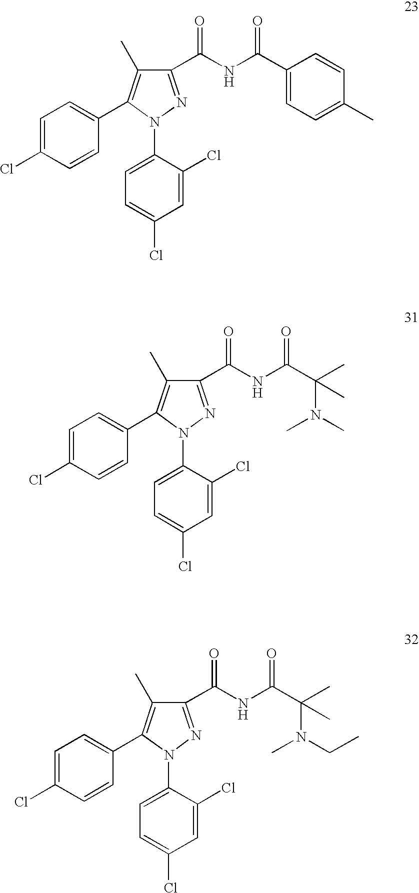 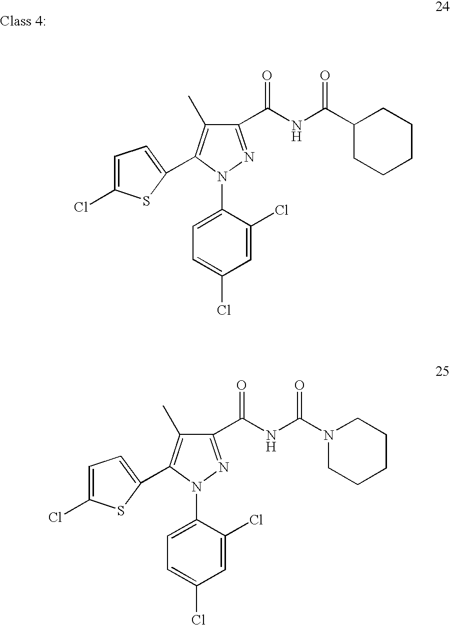 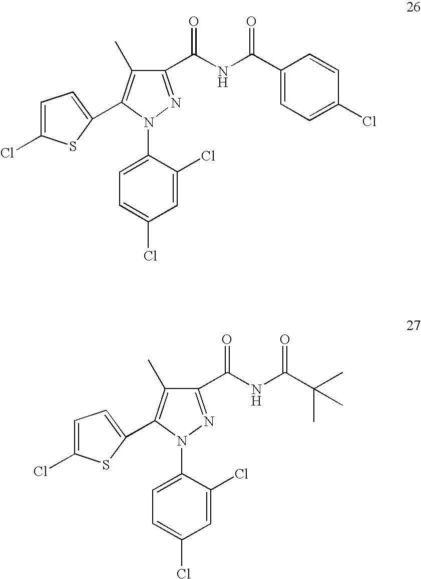 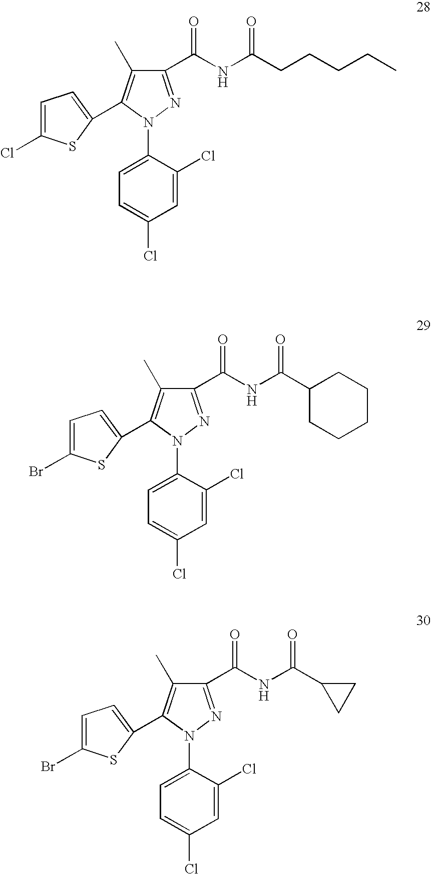
Chemical Syntheses

The procedures for synthesizing compounds 7-16 are illustrated in Scheme 1, shown below, using compounds 7 as an example. The procedures for synthesizing compounds 17-32 are illustrated in Scheme 2, also shown below, using compound 17 as an example.

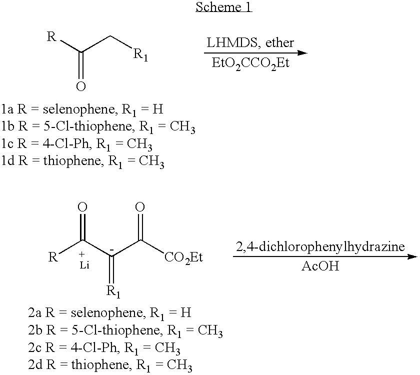

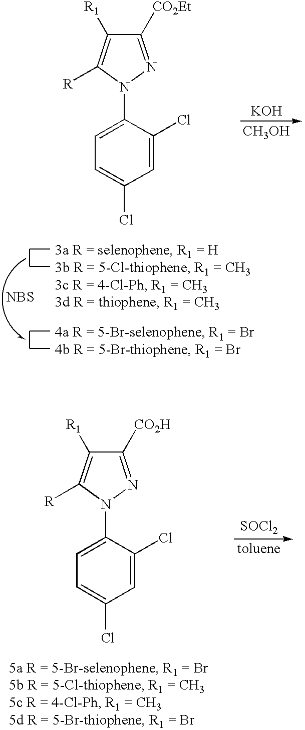

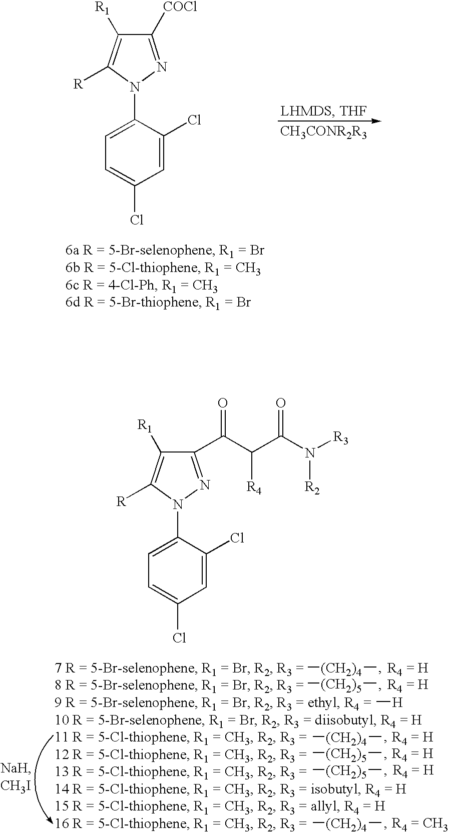

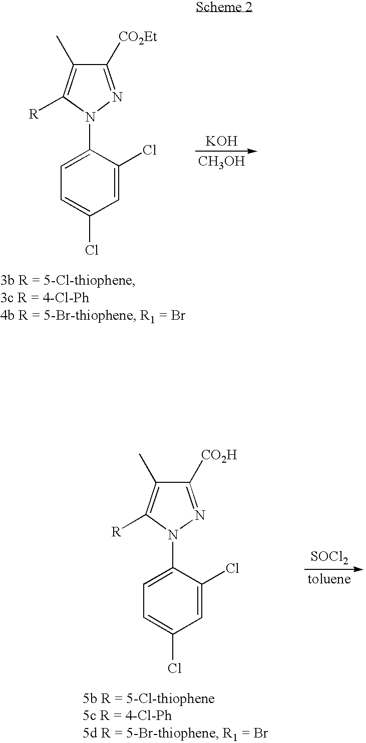

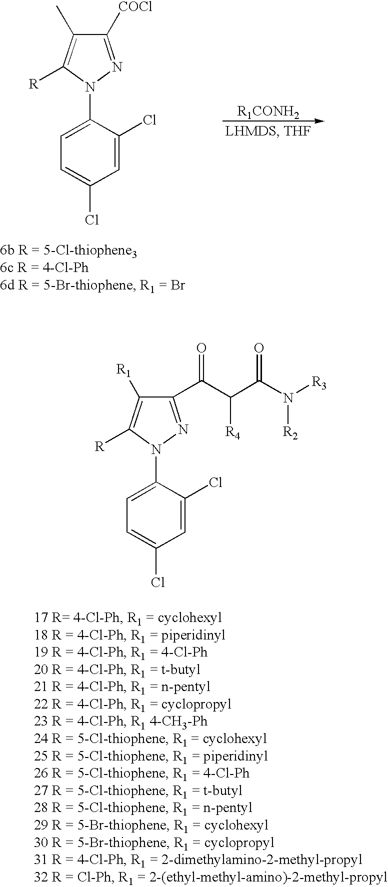

Intermediates 1a-1d are either commercially available or can be prepared according to known methods. Syntheses of intermediates 2a-2d, 3a-3d, 4a, 4b, and 5a-5d are described in 1.1-1.14 below. Syntheses of compounds 7-16 are described in 1.15-1.24 below. Synthesis of compounds 17-32 are described in 2.1-2.16 below.

### 1.1 Lithium salt of ethyl 2,4-dioxo-4-(selenophen-2-yl)-butanoate (2a)

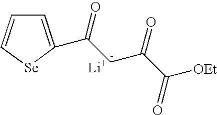

To a magnetically stirred solution of lithium bis(trimethylsilyl)amide (20.3 mL, 20.35 mmol) in diethyl ether (40 mL) was added a solution of 1-(selenophene-2-yl)ethanone 1a (3.2 g, 18.49 mmol) in diethyl ether (15 mL) at −78° C. After the mixture was stirred at the same temperature for additional 45 min, diethyl oxalate (3.0 mL, 22.19 mmol) was added to the mixture. The reaction mixture was allowed to warm to room temperature and stirred for 16 h. The precipitate was filtered, washed with diethyl ether, and dried under vacuum to afford the lithium salt 2a (3.5 g, 68%).

### 1.2 Lithium Salt of Ethyl 3-methyl-2,4-dioxo-4-(5-chlorothiophen-2-yl)-butanoate (2b)

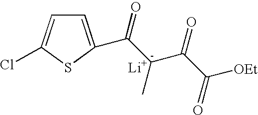

Compound 2b was synthesized from 1-(5-chlorothiophen-2-yl)-propan-1-one 1b (3.0 g, 21.39 mmol) and diethyl oxalate (3.5 mL, 25.66 mmol) according to the procedure described in 1.1 at the yield of 62% (3.2 g).

### 1.3 Lithium Salt of Ethyl 2,4-dioxo-3-methyl-4-(4-chlorophenyl)butanonte (2c)

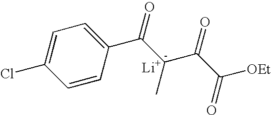

Compound 2c was synthesized from t-(4-chlorophenyl)-propan-1-one 1c (12.4 g, 73.80 mmol) and diethyl oxalate (12 mL, 89.16 mmol) according to the procedure described in 1.1 at the yield of 65% (13.2 g).

### 1.4 Lithium Salt of Ethyl 2,4-dioxo-3-methyl-4-thiophen-2-yl-butanonate (2d)

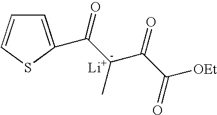

Compound 2d was synthesized from 1-(thiophen-2-yl)-propan-1-one 1d (2.6 g, 18.49 mmol) and diethyl oxalate (3.0 mL, 22.19 mmol) according to the procedure described in 1.1 at the yield of 65% (2.8 g).

### 1.5 1-(2,4-dichlorophenyl)-5-selenophene-2-yl-1H-pyrazole-3-carboxylic Acid Ethyl Ester (3a)

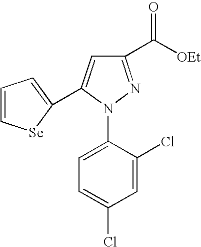

To a magnetically stirred solution of lithium salt 2a (3.5 g, 12.56 mmol) in (40 mL) of ethanol was added 2,4-dichlorophenylhydrazine hydrochloride (2.9 g, 13.82 mmol) in one portion at room temperature. The resulting mixture was stirred at room temperature for 20 h. The precipitate was filtered, washed with ethanol and diethyl ether, and then dried under vacuum to give a light yellow solid (4.0 g, 74%). This solid was dissolved in acetic acid (30 mL) and heated under reflux for 24 h. The reaction mixture was poured into ice water and extracted with ethyl acetate. The combined extracts were washed with water, saturated aqueous sodium bicarbonate, and brine, dried over anhydrous sodium sulfate, filtered, and evaporated. Purification by flash column chromatography on silica gel with n-hexane/ethyl acetate (9:1) gave ester 3a (3.0 g, 78%) as a white solid.

### 1.6 5-(5-Chlorothiophen-2-yl)-1-(2,4-dichlorophenyl)-4-methyl-1H-pyrazole-3-carboxylic Acid Ethyl Ester (3b)

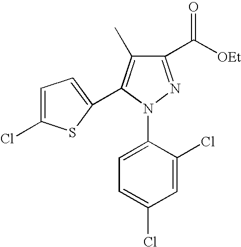

Compound 3b was synthesized from lithium salt 2b (3.2 g, 12.94 mmol) and 2,4-dichlorophenylhydrazine hydrochloride (3.0 g, 14.23 mmol) in a manner similar to that described in 1.5 as a white solid at the yield of 52% (2.7 g).

### 1.7 5-(4-Chloro-phenyl)-1-(2,4-Dichlorophenyl)-1H-pyrazole-3-carboxylic Acid Ethyl Ester (3c)

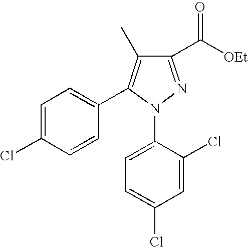

Compound 3c was synthesized from lithium salt 2c (13.2 g, 48.18 mmol) and 2,4-dichlorophenylhydrazine hydrochloride (11.3 g, 52.99 mmol) in a manner similar to that described in 1.5 as a white solid at the yield of 50% (10.8 g).

### 1.8 1-(2,4-dichlorophenyl)-4-methyl-5-thiophen-2-yl-1H-pyrazole-3-carboxylic Acid Ethyl Ester (3d)

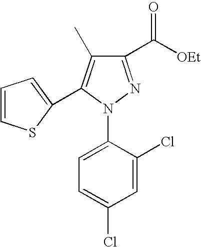

Compound 3d was synthesized from lithium salt 2d (2.8 g, 11.37 mmol) and 2,4-dichlorophenylhydrazine hydrochloride (2.6 g, 12.50 mmol) in a manner similar to that described in 1.5 as a white solid at the yield of 50% (10.8 g).

### 1.9 4-Bromo-5-(5-bromoselenophen-2-yl)-1-(2,4-dichlorophenyl)-1H-pyrazole-3-carboxylic Acid Ethyl Ester (4a)

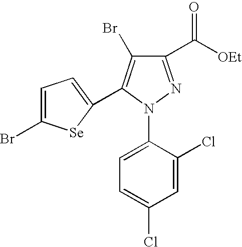

To a magnetically stirred solution of 3a (1.0 g, 2.41 mmol) in acetonitrile was added NBS (1.9 g, 7.23 mmol) in a small portions at 0° C. The resulting mixture was stirred at room temperature for 48 h. The precipitate was filtered, washed with saturated aqueous sodium sulfite and cold water, and then dried over vacuum to give compound 4a (1.9 g, 92%) as a white solid.

### 1.10 5-(5-Bromothiophen-2-yl)-1-(2,4-dichloro-phenyl)-4-methyl-1H-pyrazole-3-carboxylic Acid Ethyl Ester (4b)

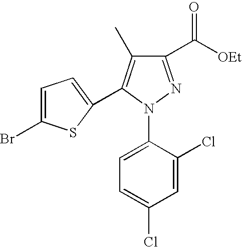

Compound 4b was synthesized from compound 3d (300 mg, 0.78 mmol) and NBS (277 mg, 1.56 mmol) in a manner similar to that described in 1.9 as a white solid at the yield of 93% (333 mg).

### 1.11 4-Bromo-5-(5-bromoselenophen-2-yl)-1-(2,4-dichlorophenyl)-1H-pyrazole-3-carboxylic Acid (5a)

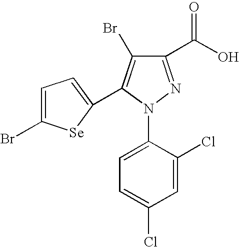

To a magnetically stirred solution of ester 4a (1.5 g, 3.62 mmol) in methanol (15 mL) was added a solution of potassium hydroxide (407 mg, 7.24 mmol) in methanol (7 mL). The mixture was heated under reflux for 3 h. The reaction mixture was cooled, poured into water, and acidified with 10% hydrochloric acid. The precipitate was filtered, washed with water, and dried under vacuum to yield the corresponding acid 5a (1.3 g, 95%) as a white solid.

### 1.12 5-(5-Chlorothiophen-2-yl)-1-(2,4-dichloro-phenyl)-4-methyl-1H-pyrazole-3-carboxylic Acid (5b)

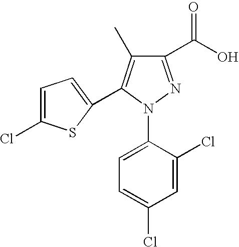

Compound 5b was synthesized from ester 3b (1.0 g, 2.40 mmol) in a manner similar to that described in 1.11 as a white solid at the yield of 95% (882 mg).

### 1.13 5-(4-Chloro-phenyl)-1-(2,4-Dichlorophenyl)-1H-pyrazole-3-carboxylic Acid (5c)

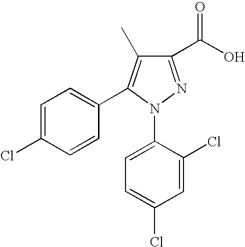

Compound 5c was synthesized from ester 3c (6.2 g, 15.07 mmol) in a manner similar to that described in 1.11 as a white solid at the yield of 97% (5.6 g).

### 1.14 5-(5-Bromothiophen-2-yl)-1-(2,4-dichlorophenyl)-4-methyl-1H-pyrazole-3-carboxylic Acid Ethyl Ester (5d)

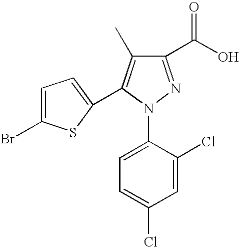

Compound 5d was synthesized from ester 4b (330 mg, 0.71 mmol) in a manner similar to that described in 1.11 as a white solid at the yield of 95% (294 mg).

### 1.15 1-[4-Bromo-5-(5-bromoselenophen-2-yl)-1-(2,4-dichlorophenyl)-1H-pyrazol-3-yl]-3-pyrrolidin-1-yl-propane-1,3-dione (7)

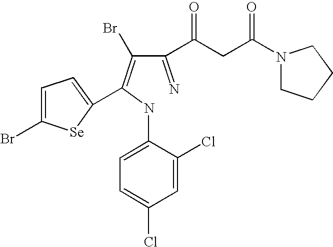

A solution of the acid 5a (60 mg, 0.11 mmol) and thionyl chloride (0.1 mL, 1.36 mmol) in toluene (5 mL) was reflux for 3 h. Solvent was evaporated under reduced pressure, and gave the crude carboxylic chloride (56 mg, 90%) as a light solid. A solution of 1-pyrrolidin-1-yl-ethanone (25 mg, 0.22 mmol) in THF was added lithium bis(trimethylsilyl)amide (0.3 mL, 0.3 mmol) at −78° C. After the mixture was stirred at the same temperature for additional 50 min, the above crude carboxylic chloride was added to the mixture and kept stirred for 2 h. The reaction was quenched with water and the aqueous layer was separated and extracted with ethyl acetate (2×10 mL). The combined extracts were washed with brine, dried over anhydrous sodium sulfate, filtered, and evaporated. Flash column chromatography of the crude product on silica gel with n-hexane/ethyl acetate (2:1) gave carboxamide 7 (39 mg, 55%) as a white solid. ¹H-NMR (CDCl₃, ppm): 7.54 (brs, 1H), 7.50 (brs, 1H), 7.41-7.39 (m, 2H), 7.16 (d, 1H), 6.98 (d, 1H), 6.05 (s, 1H), 3.59-3.46 (m, 4H), 2.02-1.85 (m, 4H), 1.33-1.25 (m, 2H), ESMS 637.8 (M+1).

### 1.16 1-[4-Bromo-5-(5-bromoselenophen-2-yl)-1-(2,4-dichlorophenyl)-1H-pyrazol-3-yl]-3-piperidin-1-yl-propane-1,3-dione (8)

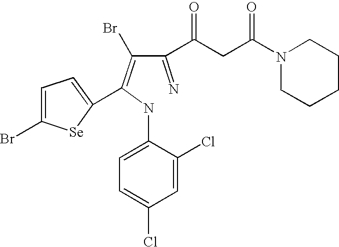

In a manner similar to that described in 1.15, treatment of crude 1-(2,4-dichlorophenyl)-4-bromo-5-(5-bromoselenophen-2-yl-1H-pyrazole-3-carboxylic chloride (60 mg, 0.11 mmol) with 1-piperidin-1-yl-ethanone (30 mg, 0.23 mmol) and lithium bis(trimethylsilyl)amide (0.3 mL, 0.27 mmol) gave compound 8 (25 mg, 36%) as a white solid.: ¹H-NMR (CDCl₃, ppm): 7.55 (brs, 1H), 7.43-7.38 (m, 2H), 7.17 (d, 1H), 6.98 (d, 1H), 6.21 (s, 1H), 4.16 (s, 2H), 3.58 (t, 2H), 3.37 (t, 2H), 1.72-1.50 (m, 4H), 1.30-1.21 (m, 2H); ESMS 651.8 (M+1).

### 1.17 3-[4-Bromo-5-(5-bromoselenophen-2-yl)-1-(2,4-dichlorophenyl)-1H-pyrazol-3-yl]-N,N-diethyl-3-oxo-propionamide (9)

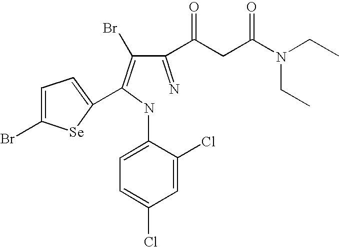

In a manner similar to that described in 1.15, treatment of crude 1-(2,4-dichlorophenyl)-4-bromo-5-(5-bromoselenophen-2-yl-1H-pyrazole-3-carboxylic chloride (60 mg, 0.11 mmol) with N,N-Diethyl-acetamide (25 mg, 0.22 mmol) and lithium bis(trimethylsilyl)amide (0.3 mL, 0.3 mmol) gave compound 9 (30 mg, 43%) as a white solid. ¹H-NMR (CDCl₃, ppm): 7.54-7.50 (m, 1H), 7.43-7.39 (m, 2H), 7.16 (d, 1H), 6.99-6.97 (m, 1H), 6.15 (s, 1H), 3.48-3.28 (m, 4H), 1.28-1.11 (m, 6H), ESMS 639.7 (M+1).

### 1.18 3-[4-Bromo-5-(5-bromoselenophen-2-yl)-1-(2,4-dichlorophenyl)-1H-pyrazol-3-yl]-N,N-diisobutyl-3-oxo-propionamide (10)

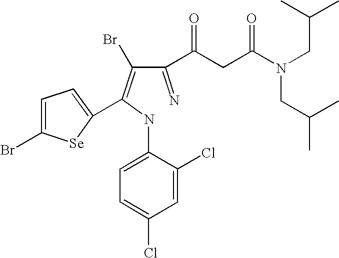

In a manner similar to that described in 1.15, treatment of crude 1-(2,4-dichlorophen-yl)-4-bromo-5-(5-bromoselenophen-2-yl-1H-pyrazole-3-carboxylic chloride (60 mg, 0.11 mmol) with N,N-Diisobutyl-acetamide (31 mg, 0.22 mmol) and lithium bis(trimethylsilyl)amide (0.3 mL, 0.3 mmol) gave compound 10 (45 mg, 61%) as a white solid. ¹H-NMR (CDCl₃, ppm): 7.46 (brs, 1H), 7.32 (brs, 2H), 7.09 (d, 1H), 6.91 (d, 1H), 6.15 (b, 1H), 3.20-3.04 (m, 4H), 1.98-1.94 (m, 2H), 0.91-0.70 (m, 12H), ESMS 695.8 (M+1).

### 1.19 1-[5-(5-Chloro-thiophen-2-yl)-1-(2,4-dichloro-phenyl)-4-methyl-1H-pyrazol-3-yl-3-pyrrolidin-1-yl-propane-1,3-dione (11)

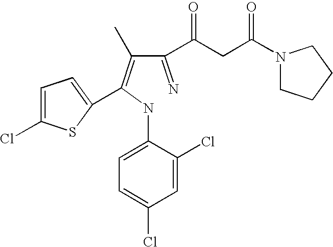

In a manner similar to that described in 1.15, treatment of crude 5-(5-chloro-thiophen-2-yl)-1-(2,4-dichloro-phenyl)-4-methyl-1H-pyrazole-3-carbonyl chloride (100 mg, 0.26 mmol) with 1-pyrrolidin-1-yl-ethanone (59 mg, 0.52 mmol) and lithium bis(trimethylsilyl)amide (0.7 mL, 0.7 mmol) gave compound 11 (44 mg, 35%) as a white solid. ¹H-NMR (CDCl₃, ppm): 7.51 (brs, 1H), 7.47 (m, 2H), 6.82 (d, 1H), 6.66 (d, 11H), 5.84 (s, 1H), 4.11 (s, 2H), 2.43-3.47 (m, 4H), 2.41 (s, 3H), 2.38 (s, 3H), 2.00-1.85 (m, 4H); ESMS 482.1 (M+1).

### 1.20 1-[5-(5-Chlorothiophen-2-yl)-1-(2,4-dichlorophenyl)-4-methyl-1H-pyrazol-3-yl]-3-piperidin-1-yl-propane-1,3-dione (12)

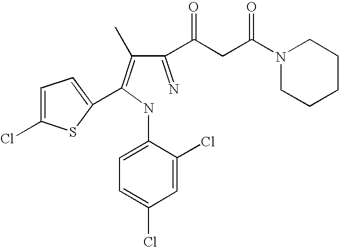

In a manner similar to that described in 1.15, treatment of crude 5-(5-chlorothiophen-2-yl)-1-(2,4-dichlorophenyl)-4-methyl-1H-pyrazole-3-carbonyl chloride (100 mg, 0.26 mmol) with 1-piperidin-1-yl-ethanone (66 mg, 0.52 mmol) and lithium bis(trimethylsilyl)amide (0.7 mL, 0.7 mmol) gave compound 12 (53 mg, 41%) as a white solid. ¹H-NMR (CDCl₃, ppm): 7.51-7.50 (m, 1H), 7.36-7.34 (m, 2H), 6.81 (d, 1H), 6.65 (d, 1H), 6.04 (s, 1H), 4.18 (s, 2H), 3.61-3.58 (m, 2H), 3.40-3.71 (m, 2H), 2.41 (s, 3H), 2.39 (s, 3H), 1.63-1.57 (m, 4H), 1.28-1.26 (m, 2H), ESMS 496.1 (M+1).

### 1.21 1-Azepan-1-yl-3-[5-(5-chlorothiophen-2-yl)-1-(2,4-dichlorophenyl)-4-methyl-1H-pyrazol-3-yl]-propane-1,3-dione (13)

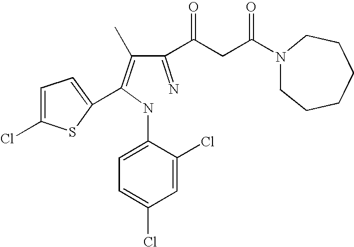

In a manner similar to that described in 1.15, treatment of crude 5-(5-chloro-thiophen-2-yl)-1-(2,4-dichlorophenyl)-4-methyl-1H-pyrazole-3-carbonyl chloride (100 mg, 0.25 mmol) with 1-azepan-1-yl-ethanone (53 μL, 0.50 mmol) and lithium bis(trimethylsilyl)amide (0.55 mL, 0.55 mmol) gave compound 13 (104.6 mg, 82%) as a white solid. ¹H-NMR (CDCl₃, ppm): 7.46 (brs, 1H), 7.40-7.26 (m, 2H), 6.77 (d, 1H), 6.62 (d, 1H), 4.20-4.02 (m, 2H), 3.51 (t, 2H), 3.41 (t, 2H), 2.38 (s, 3H), 1.80-1.60 (m, 4H), 1.60-1.40 (m, 4H); ESMS 510.1 (M+1).

### 1.22 3-[5-(5-Chlorothiophen-2-yl)-1-(2,4-dichlorophenyl)-4-methyl-1H-pyrazol-3-yl]-N,N-diisobutyl-3-oxo-propionamide (14)

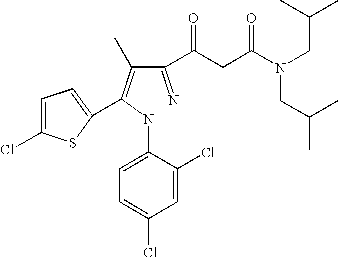

In a manner similar to that described in 1.15, treatment of crude 5-(5-chlorothiophen-2-yl)-1-(2,4-dichlorophenyl)-4-methyl-1H-pyrazole-3-carbonyl chloride (100 mg, 0.25 mmol) with N,N-Diisobutyl-acetamide (55.0 μL, 0.50 mmol) and lithium bis(trimethylsilyl)amide (0.55 mL, 0.55 mmol) gave compound 14 (113.7 mg, 84%) as a white solid. ¹H-NMR (CDCl₃, ppm): 7.49 (brs, 1H), 7.40-7.26 (m, 2H), 6.80 (d, 1H), 6.64 (d, 1H), 4.20-4.02 (m, 2H), 3.20 (d, 2H), 3.09 (d, 2H), 2.41 (s, 3H), 2.05-1.94 (m, 2H), 0.88 (d, 3H), 0.88 (d, 3H); ESMS 540.1 (M+1).

### 1.23 N,N-Diallyl-3-[5-(5-chlorothiophen-2-yl)-1-(2,4-dichlorophenyl)-4-methyl-1H-pyrazol-3-yl]-3-oxo-propionamide (15)

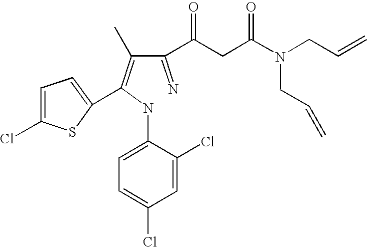

In a manner similar to that described in 1.15, treatment of crude 5-(5-chlorothiophen-2-yl)-1-(2,4-dichlorophenyl)-4-methyl-1H-pyrazole-3-carbonyl chloride (100 mg, 0.25 mmol) with N,N-diallyl-acetamide (52.0 μL, 0.50 mmol) and lithium bis(trimethylsilyl)amide (0.55 mL, 0.55 mmol) gave compound 15 (99.1 mg, 78%) as a white solid. ¹H-NMR (CDCl₃, ppm): 7.50 (brs, 1H), 7.40-7.28 (m, 2H), 6.82 (d, 1H), 6.65 (d, 1H), 5.90-5.70 (m, 2H), 5.30-5.10 (m, 4H), 4.20-4.10 (m, 2H), 4.02 (d, 2H), 3.92 (d, 2H), 2.41 (s, 3H); ESMS 508.0 (M+1).

### 1.24 1-[5-(5-Chlorothiophen-2-yl)-1-(2,4-dichlorophenyl)-4-methyl-1H-pyrazol-3-yl]-2-methyl-3-pyrrolidin-1-yl-propane-1,3-dione (16)

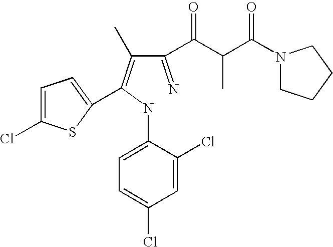

To a solution of NaH (8.3 mg, 0.2 mmol) in EtOH (2 mL) was added a solution of compound 11 (20 mg) in EtOH (2 mL) dropwise. The reaction mixture was stirred at room temperature. After 1 h, CH₃I (0.1 mL, 1.6 mmol) was added gave compound 16 (10 mg, 49%) as a white solid. ¹H-NMR (CDCl₃, ppm): 7.45 (d, 1H), 7.30-7.14 (m, 2H), 6.74 (d, 1H), 6.56 (d, 1H), 4.67-4.46 (m, 1H), 3.68-3.56 (m, 1H), 3.46-3.32 (m, 2H), 2.33 (s, 3H), 1.88-1.61 (m, 3H), 1.36 (d, 1H); ESMS 496.1 (M+1).

### 2.1 N-(Cyclohexanecarbonyl)-1-(2,4-dichlorophenyl)-4-methyl-5-(4-chlorophenyl)-1H-pyrazole-3-carboxamide (17)

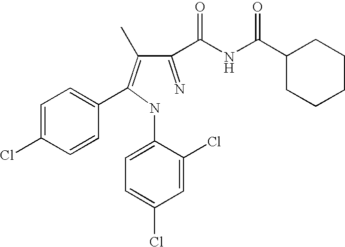

A solution of the acid 5c (80 mg, 0.21 mmol) and thionyl chloride (0.88 mL, 1.2 mmol) in toluene (5 mL) was reflux for 3 h. Solvent was evaporated under reduced pressure, and gave the crude carboxylic chloride (56 mg, 90%) as a light solid. To a solution of cyclohexanecarboxamide (0.06 g, 0.44 mmol) in THF (3 mL) was added lithium bis(trimethylsilyl)amide (0.48 mL, 0.53 mmol) at −78° C. After the mixture was stirred at the same temperature for additional 50 min, a solution of the above carboxylic chloride in THF (5 ml) was added dropwise to the mixture. The reaction mixture was allowed to warm to −10° C. and stirred for additional 2 h. The reaction was quenched with water and subjected to extraction with ethyl acetate (3×15 mL). The combined extracts were washed with brine, dried over anhydrous magnesium sulfate, filtered, and evaporated. Flash column chromatography on silica gel with n-hexane/ethyl acetate (4:1) gave carboxamide 17 (99 mg, 97% yield) as a white solid. 9.33 (brs, 1H), 7.44 (d, 1H), 7.34-7.25 (m, 4H), 7.08 (d, 2H), 3.28-3.21 (m, 1H), 2.38 (s, 3H), 2.01 (d, 2H), 1.83 (d, 2H), 1.73 (d, 1H), 1.54-1.32 (m, 5H); ESMS 512.2 (M+23).

### 2.2 N-(piperidine-1-carbonyl)-1-(2,4-dichlorophenyl)-4-methyl-5-(4-chlorophenyl)-1H-pyrazole-3-carboxamide (18)

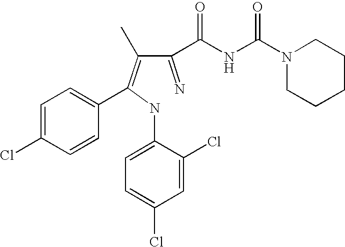

In a manner similar to that described in 2.1, treatment of crude 5-(4-chlorophenyl)-1-(2,4-dichlorophenyl)-4-methyl-1H-pyrazole-3-carbonyl chloride (104 mg, 0.26 mmol) with 1-piperidinecarboxamide (74 mg, 0.58 mmol) and lithium bis(trimethylsilyl)amide (0.64 mL, 0.70 mmol,) gave compound 18 (134 mg, 98%) as a white solid. ¹H-NMR (CDCl₃, ppm): 8.60 (br, 1H), 7.42 (s, 1H), 7.32-7.26 (m, 4H), 7.08 (d, 2H), 3.58-3.42 (m, 4H), 2.35 (s, 3H), 1.72-1.58 (m, 6H); ESMS 491.2 (M+1).

### 2.3 N-(4-chloro-benzoyl)-1-(2,4-dichlorophenyl)-4-methyl-5-(4-chlorophenyl)-1H-pyrazole-3-carboxamide (19)

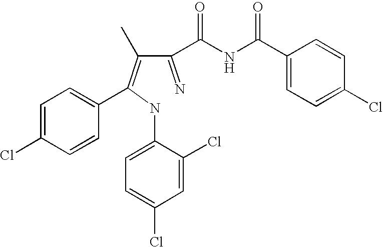

In a manner similar to that described in 2.1, treatment of crude 5-(4-chlorophenyl)-1-(2,4-dichlorophenyl)-4-methyl-1H-pyrazole-3-carbonyl chloride (55 mg, 0.12 mmol) with 4-Chlorobenzamide (47 mg, 0.30 mmol) and lithium bis(trimethylsilyl)amide (0.34 mL, 0.37 mmol) gave compound 19 (71 mg, 95%) as a white solid. ¹H-NMR (CDCl₃, ppm): 10.10 (br, 1H), 7.84 (d, 2H), 7.50-7.42 (m, 3H), 7.38-7.28 (m, 4H), 7.10 (d, 2H), 2.39 (s, 3H); ESMS 491.2 (M+1).

### 2.4 N-(2,2-dimethyl-propionyl)-1-(2,4-dichlorophenyl)-4-methyl-5-(4-chlorophenyl)-1H-pyrazole-3-carboxamide (20)

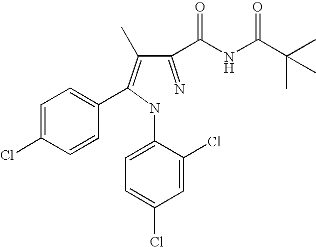

In a manner similar to that described in 2.1, treatment of crude 5-(4-chlorophenyl)-1-(2,4-dichlorophenyl)-4-methyl-1H-pyrazole-3-carbonyl chloride (55 mg, 0.12 mmol) with trimethylacetamide (31 mg, 0.30 mmol) and lithium bis(trimethylsilyl)amide (0.34 mL, 0.37 mmol) gave compound 20 (68 mg, 99%) as a white solid. ¹H-NMR (CDCl₃, ppm): 9.81 (br, 1H), 7.46 (d, 1H), 7.34-7.25 (m, 4H), 7.08 (d, 2H), 2.37 (s, 3H), 1.29 (s, 9H); ESMS 464.0 (M+1).

### 2.5 N-(Hexanoyl)-1-(2,4-dichlorophenyl)-4-methyl-5-(4-chlorophenyl)-1H-pyrazole-3-carboxamide (21)

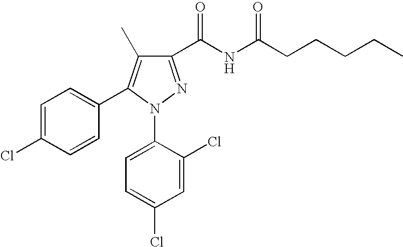

In a manner similar to that described in 2.1, treatment of crude 5-(4-chlorophenyl)-1-(2,4-dichlorophenyl)-4-methyl-1H-pyrazole-3-carbonyl chloride (55 mg, 0.12 mmol) with hexanoamide (35 mg, 0.30 mmol) and lithium bis(trimethylsilyl) amide (0.34 mL, 0.37 mmol) gave compound 21 (33 mg, 48%) as a white solid. ¹H-NMR (CDCl₃, ppm): 9.36 (brs, 1H), 7.45 (d, 1H), 7.35-7.24 (m, 4H), 7.08 (d, 2H), 2.96 (t, 2H), 2.37 (s, 3H), 1.78-1.65 (m, 2H), 1.45-1.31 (m, 4H), 0.91 (t, 2H); ESMS 478.0 (M+1).

### 2.6 N-(Cyclopropanecarbonyl)-1-(2,4-dichlorophenyl)-4-methyl-5-(4-chlorophenyl)-1H-pyrazole-3-carboxamide (22)

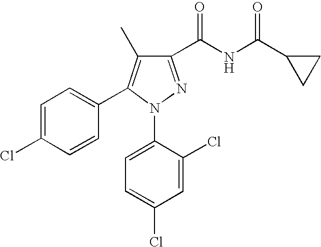

In a manner similar to that described in 2.1, treatment of crude 5-(4-chlorophenyl)-1-(2,4-dichloro-phenyl)-4-methyl-1H-pyrazole-3-carbonyl chloride (55 mg, 0.12 mmol) with cyclopropanecarboxamide (33 mg, 0.39 mmol) and lithium bis-(trimethylsilyl) amide (0.42 mL, 0.46 mmol) gave compound 22 (57 mg, 96%) as a white solid. ¹H-NMR (CDCl₃, ppm): 9.43 (brs, 1H), 7.44 (d, 1H), 7.34-7.26 (m, 4H), 7.09 (d, 2H), 3.03-2.97 (m, 1H), 2.39 (s, 3H), 1.23-1.18 (m, 2H), 1.05-0.94 (m, 2H); ESMS 470.0 (M+23).

### 2.7 N-(4-methyl-benzoyl)-1-(2,4-dichlorophenyl)-4-methyl-5-(4-chlorophenyl)-1H-pyrazole-3-carboxamide (23)

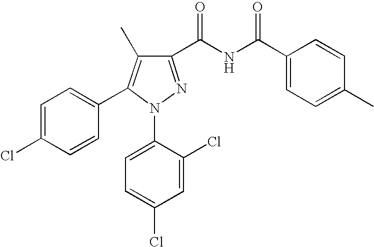

In a manner similar to that described in 2.1, treatment of crude 5-(4-chlorophenyl)-1-(2,4-dichlorophenyl)-4-methyl-1H-pyrazole-3-carbonyl chloride (55 mg, 0.12 mmol) with p-Toluamide (53 mg, 0.39 mmol) and lithium bis(trimethylsilyl) amide (0.42 mL, 0.46 mmol) gave compound 23 (62 mg, 94%) as a white solid. ¹H-NMR (CDCl₃, ppm): 10.15 (br, 1H), 7.80 (d, 2H), 7.46 (s, 1H), 7.38-7.23 (m, 6H), 7.10 (d, 2H), 2.40 (s, 3H), 2.40 (s, 3H); ESMS 520.0 (M+23).

### 2.8 N-(Cyclohexanecarbonyl)-1-(2,4-dichlorophenyl)-4-methyl-5-(5-chlorothiophen-2-yl)-1H-pyrazole-3-carboxamide (24)

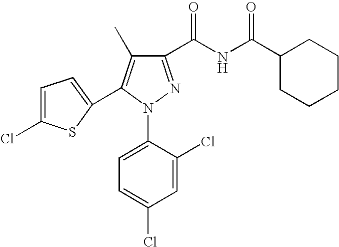

In a manner similar to that described in 2.1, treatment of crude 5-(5-chlorothiophen-2-yl)-1-(2,4-dichlorophenyl)-4-methyl-1H-pyrazole-3-carbonyl chloride (57 mg, 0.14 mmol) with cyclohexanecarboxamide (38 mg, 0.30 mmol) and lithium bis(trimethylsilyl)amide (0.34 mL, 0.37 mmol) gave compound 24 (68 mg, 96%) as a white solid. ¹H-NMR (CDCl₃, ppm): 9.29 (br, 1H), 7.52 (d, 1H), 7.40-7.27 (m, 2H), 6.84 (d, 1H), 6.69 (d, 1H), 3.26-3.18 (m, 1H), 2.47 (s, 3H), 2.00 (d, 2H), 1.83 (d, 2H), 1.72 (d, 1H), 1.54-1.19 (m, 5H); ESMS 518.0 (M+23).

### 2.9 N-(piperidine-1-carbonyl)-1-(2,4-dichlorophenyl)-4-methyl-5-(5-chlorothiophen-2-yl)-1H-pyrazole-3-carboxamide (25)

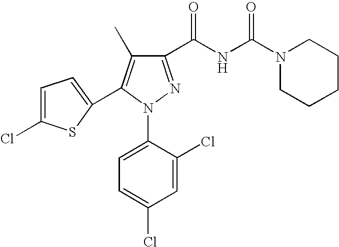

In a manner similar to that described in 2.1, treatment of crude 5-(5-chlorothiophen-2-yl)-1-(2,4-dichlorophenyl)-4-methyl-1H-pyrazole-3-carbonyl chloride (57 mg, 0.14 mmol) with 1-piperidinecarboxamide (38 mg, 0.30 mmol) and lithium bis(trimethylsilyl)amide (0.34 mL, 0.37 mmol) gave compound 25 (66 mg, 94%) as a white solid. ¹H-NMR (CDCl₃, ppm): 8.47 (brs, 1H), 7.51 (d, 1H), 7.40-7.26 (m, 2H), 6.83 (d, 1H), 6.69 (d, 1H), 3.58-3.42 (m, 4H), 2.45 (s, 3H), 1.69-1.56 (m, 6H); ESMS 497.3 (M+1).

### 2.10 N-(4-chloro-benzoyl)-1-(2,4-dichlorophenyl)-4-methyl-5-(5-chlorothiophen-2-yl)-1H-pyrazole-3-carboxamide (26)

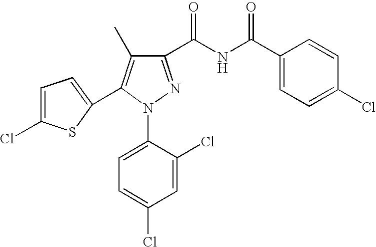

In a manner similar to that described in 2.1, treatment of crude 5-(5-chlorothiophen-2-yl)-1-(2,4-dichloro-phenyl)-4-methyl-1H-pyrazole-3-carbonyl chloride (57 mg, 0.14 mmol) with 4-chlorobenzamide (47 mg, 0.30 mmol) and lithium bis(trimethyl-silyl)amide (0.34 mL, 0.37 mmol) gave compound 26 (68 mg, 99%) as a white solid. ¹H-NMR (CDCl₃, ppm): 10.05 (brs, 1H), 7.82 (d, 2H), 7.54 (d, 1H), 7.47-7.35 (m, 4H), 6.85 (d, 1H), 6.72 (d, 1H), 2.48 (s, 3H); ESMS 546.0 (M+23).

### 2.11 N-(2,2-dimethyl-propionyl)-1-(2,4-dichlorophenyl)-4-methyl-5-(5-chlorothiophen-2-yl)-1H-pyrazole-3-carboxamide (27)

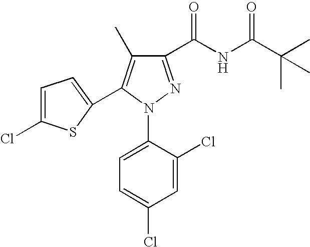

In a manner similar to that described in 2.1, treatment of crude 5-(5-chlorothiophen-2-yl)-1-(2,4-dichlorophenyl)-4-methyl-1H-pyrazole-3-carbonyl chloride (62 mg, 0.15 mmol) with trimethylacetamide (33 mg, 0.33 mmol) and lithium bis(trimethylsilyl)amide (0.36 mL, 0.39 mmol) gave compound 27 (73 mg, 99%) as a white solid. ¹H-NMR (CDCl₃, ppm): 9.76 (brs, 1H), 7.53 (d, 1H), 7.41-7.32 (m, 2H), 6.84 (d, 1H), 6.69 (d, 1H), 2.46 (s, 3H), 1.26 (s, 9H); ESMS 470.0 (M+1).

### 2.12 N-(hexanoyl)-1-(2,4-dichlorophenyl)-4-methyl-5-(5-chlorothiophen-2-yl)-1H-pyrazole-3-carboxamide (28)

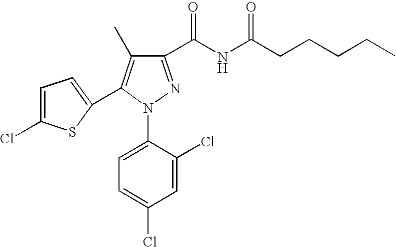

In a manner similar to that described in 2.1, treatment of crude 5-(5-chlorothiophen-2-yl)-1-(2,4-dichlorophenyl)-4-methyl-1H-pyrazole-3-carbonyl chloride (62 mg, 0.15 mmol) with hexanoamide (38 mg, 0.33 mmol) and lithium bis(trimethylsilyl) amide (0.36 mL, 0.39 mmol) gave compound 28 (63 mg, 85%) as a white solid. ¹H-NMR (CDCl₃, ppm): 9.32 (brs, 1H), 7.52 (d, 1H), 7.39-7.30 (m, 2H), 6.84 (d, 1H), 6.69 (d, 1H), 2.94 (t, 2H), 2.46 (s, 3H), 1.77-1.67 (m, 2H), 1.43-1.30 (m, 4H), 0.91 (t, 2H); ESMS 506.0 (M+23).

### 2.13 N-(Cyclohexanecarbonyl)-1-(2,4-dichlorophenyl)-4-methyl-5-(5-bromothiophen-2-yl)-1H-pyrazole-3-carboxamide (29)

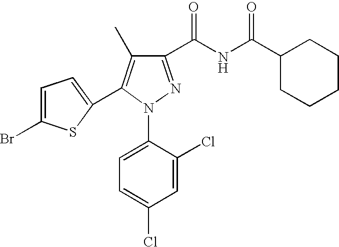

In a manner similar to that described in 2.1, treatment of crude 5-(5-bromothiophen-2-yl)-1-(2,4-dichloro-phenyl)-4-methyl-1H-pyrazole-3-carbonyl chloride (62 mg, 0.15 mmol) with cyclohexanecarboxamide (37 mg, 0.29 mmol) and lithium bis(trimethylsilyl) amide (0.32 mL, 0.35 mmol) gave compound 29 (65 mg, 88%) as a white solid. ¹H-NMR (CDCl₃, ppm): 9.28 (brs, 1H), 7.52 (d, 1H), 7.40-7.25 (m, 2H), 6.97 (d, 1H), 6.66 (d, 1H), 3.27-3.15 (m, 1H), 2.47 (s, 3H), 1.99 (d, 2H), 1.83 (d, 2H), 1.73 (d, 1H), 1.55-1.20 (m, 5H); ESMS 540.1 (M+1).

### 2.14 N-(Cyclopropanecarbonyl)-1-(2,4-dichlorophenyl)-4-methyl-5-(5-bromothiophen-2-yl)-1H-pyrazole-3-carboxamide (30)

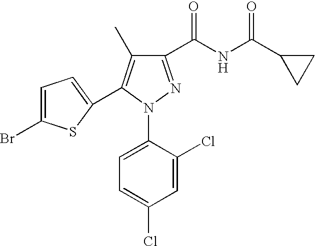

In a manner similar to that described in 2.1, treatment of crude 5-(5-bromothiophen-2-yl)-1-(2,4-dichlorophenyl)-4-methyl-1H-pyrazole-3-carbonyl chloride (62 mg, 0.15 mmol) with cyclopropanecarboxamide (25 mg, 0.29 mmol) and lithium bis-(trimethylsilyl) amide (0.32 mL, 0.35 mmol) gave compound 30 (66 mg, 97%) as a white solid. ¹H-NMR (CDCl₃, ppm): 9.39 (br, 1H), 7.52 (d, 1H), 7.40-7.25 (m, 2H), 6.98 (d, 1H), 6.67 (d, 1H), 3.05-2.92 (m, 1H), 2.48 (s, 3H), 1.24-1.15 (m, 2H), 1.07-0.95 (m, 2H); ESMS 498.0 (M+1).

### 2.15 N-(2-dimethylamino-2-methyl-propionyl)-1-(2,4-dichlorophenyl)-4-methyl-5-(4-chlorophenyl)-1H-pyrazole-3-carboxamide (31)

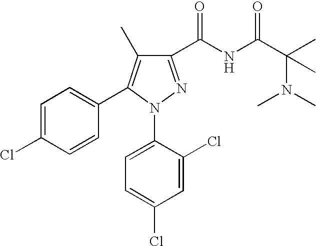

In a manner similar to that described in 2.1, treatment of crude 5-(4-chlorophenyl)-1-(2,4-dichlorophenyl)-4-methyl-1H-pyrazole-3-carbonyl chloride (60 mg, 0.15 mmol) with 2-dimethylamino-2-methyl-propionamide (63 mg, 0.49 mmol) and lithium bis(trimethylsilyl) amide (0.53 mL, 0.58 mmol) gave compound 31 (59 mg, 80%) as a white solid. ¹H-NMR (CDCl₃, ppm): 11.37 (br, 1H), 7.46 (d, 1H), 7.35-7.21 (m, 4H), 7.070 (d, 2H), 2.38 (s, 3H), 2.23 (s, 6H), 1.24 (s, 6H); ESMS 493.1 (M+1).

### 2.16 N-[2-(ethyl-methyl-amino)-2-methyl-propionyl]-1-(2,4-dichlorophenyl)-4-methyl-5-(4-chlorophenyl)-1H-pyrazole-3-carboxamide (32)

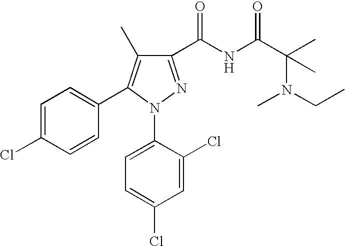

In a manner similar to that described in 2.1, treatment of crude 5-(4-chlorophenyl)-1-(2,4-dichlorophenyl)-4-methyl-1H-pyrazole-3-carbonyl chloride (55 mg, 0.12 mmol) with 2-(Ethyl-methyl-amino)-2-methyl-propionamide (56 mg, 0.39 mmol) and lithium bis(trimethylsilyl) amide (0.42 mL, 0.46 mmol) gave compound 32 (46 mg, 75%) as a white solid. ¹H-NMR (CDCl₃, ppm): 11.46 (brs, 1H), 7.45 (d, 1H), 7.33-7.24 (m, 4H), 7.09 (d, 2H), 2.38 (s, 3H), 2.33 (q, 2H), 2.20 (s, 3H), 1.25 (s, 6H), 1.07 (t, 3H); ESMS 530.0 (M+23).

Biological Assays

The affinity of test compounds of this invention toward CB1 and CB2 receptors was determined by competitive radioligand binding assays in vitro. This method differentiates the binding strength between compounds by their abilities in displacing a receptor-specific radioactive ligand. Compounds with higher affinity than the radioactive ligand displace the ligand and bind to the receptors, while compounds with no affinity or lower affinity than the radioactive ligand do not. The readings of the radioactivity retained allow further analysis of receptor binding, and assist in predictions of the pharmacological activities of the test compounds.

In the assays, CB1 receptors are either from rat brain or CB1 stably expressed cell lines, and CB2 receptors are from rat spleen or CB2 stably expressed cell lines. Male Sprague-Dawley rats weighing 175-200 g were used and housed under standard stalling conditions with food and water available ad libitum. The animals were sacrificed, and brain with cerebellum excluded and spleen were dissected from the animals. The separated brain and spleen tissues were respectively homogenized by Polytron Homogenizers in 10 volumes of ice-cold buffer A (50 mM Tris, 5 mM MgCl₂, 2.5 mM EDTA, pH 7.4, 10% sucrose) with protease inhibitors. The homogenate was centrifuged for 15 minutes at 2,000×g at 4° C. The resultant supernatant was centrifuged again for 30 minutes at 43,000×g at 4° C. The final pellet was re-suspended in buffer A and stored at −80° C. For purification of membrane-enriched fractions of CB1 or CB2 stably expressed cell lines, cells were scraped out from the culture dishes. After sonication, the membrane-enriched fractions were purified by following the same centrifugation and storing procedures. The protein concentration of the purified membrane was determined by the Bradford method as described by the manual provided by Bio-Rad Laboratories, Inc., Hercules, Calif.

During the receptor binding experiments, 0.2˜8 μg of membrane fractions were incubated with 0.75 nM [³H]CP55,940 and a test compound in the incubation buffer of 50 mM Tris-HCl, 5 mM MgCl₂, 1 mM EDTA, 0.3% BSA, pH 7.4. The non-specific binding was determined by using 1 μM of CP55,940. The mixture was incubated for 1.5 hours at 30° C. in Multiscreen microplates (Millipore, Billerica, Mass.). At the completion of the incubation, the reaction was terminated by Manifold filtration and washed with ice-cold wash buffer (50 mM Tris, pH 7.4, 0.25% BSA) four times. The radioactivity bound to the filters was measured by Topcount (Perkin Elmer Inc.). IC₅₀ values were calculated based on the concentration of the test compound required to inhibit 50% of the binding of [³H]CP55,940.

The efficacy of each test compound was determined by DELFIA GTP-binding kit (Perkin Elmer Inc., Boston, Mass.). The DELFIA GTP-binding assay is a time-resolved fluorometric assay based on GDP-GTP exchange on G-protein subunits followed by activation of a G protein-coupled receptor by its agonists. Eu-GTP was used in this assay to allow monitoring of agonist-dependent activation of G-protein. Note that stimulation of CB1 receptors by CP55,940 leads to the replacement of GDP by GTP on the α-subunit of G-protein. The resultant GTP-Gα complex represents the activated form of G-protein. Eu-GTP, a non-hydrolysable analogue of GTP, can be used to quantify the amount of activated G-protein (Peltonen et al., Eur. J. Pharmacol. (1998) 355:275).

Plasma membrane of human CB1-expressing HEK293 cells was re-suspended in an assay buffer (50 mM HEPES, pH 7.4, 100 mM NaCl, 100 μg/mL saponin, 5 mM MgCl₂, 2 μM GDP, 0.5% BSA). An aliquot of membrane was added to each well of an AcroPlate (Pall Life Sciences, Ann Arbor, Mich.). After the addition of a test compound (various concentrations in 0.1% DMSO) and CP55,940 (20 nM in the assay buffer), the assay plate was incubated in the dark at 30° C. with slow shaking for 60 minutes. Eu-GTP was added to each well and the plate was incubated for another 35 minutes at 30° C. in the dark. The assay was terminated by washing the plate four times with a wash solution provided in the assay kit. Binding of the Eu-GTP was determined based on the fluorescence signal from a Victor 2 multi-label reader. The IC₅₀ value (i.e., 50% inhibition of CP55,940-stimulated Eu-GTP binding) for each test compound was determined by a concentration-response curve using nonlinear regression (Prism; GraphPad, San Diego, Calif.).

All of the test compounds 7-32 showed IC₅₀ values between 0.1 nM and 30 μM in the CB1 receptor binding assays and/or CB2 receptor binding assays. The Eu-GTP binding assays were also conducted, and the results were comparable to those obtained from the above-mentioned radioligand binding assays.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula (I): 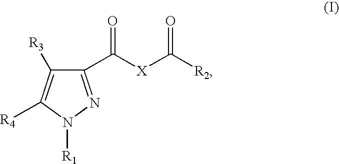
wherein
X is C(RₐR_{b}) or N(Rₐ), in which each of Rₐ and R_{b}, independently, is H, C₁-C₁₀ alkyl, C₃-C₂₀ cycloalkyl, C₁-C₂₀ heterocycloalkyl, aryl, or heteroaryl;
R₂ is H, halo, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, or NR_{c}R_{d}, in which each of R_{c} and R_{d}, independently, is H, C₁-C₁₀ alkyl, C₃-C₂₀ cycloalkyl, C₁-C₂₀ heterocycloalkyl, aryl, or heteroaryl; and
each of R₁, R₃, and R₄, independently, is H, halo, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

2. The compound of claim 1, wherein X is CH₂.

3. The compound of claim 2, wherein R₂ is C₁-C₂₀ heterocycloalkyl or NR_{c}R_{d}, in which each of R_{c} and R_{d}, independently, is H, C₁-C₁₀ alkyl, C₃-C₂₀ cycloalkyl, C₁-C₂₀ heterocycloalkyl, aryl, or heteroaryl.

4. The compound of claim 1, wherein X is NH.

5. The compound of claim 4, wherein R₂ is C₁-C₁₀ alkyl, C₃-C₂₀ cycloalkyl, C₁-C₂₀ heterocycloalkyl, or aryl.

6. The compound of claim 5, wherein R₁ is aryl substituted with halo.

7. The compound of claim 6, wherein R₁ is 2,4-dichlorophenyl.

8. The compound of claim 1, wherein R₁ is aryl substituted with halo.

9. The compound of claim 8, wherein R₁ is 2,4-dichlorophenyl.

10. The compound of claim 1, wherein R₄ is aryl or heteroaryl.

11. The compound of claim 1, wherein R₂ is C₁-C₁₀ alkyl, C₃-C₂₀ cycloalkyl, C₁-C₂₀ heterocycloalkyl, aryl, or NR_{c}R_{d}, in which each of R_{c} and R_{d}, independently, is H, C₁-C₁₀ alkyl, C₃-C₂₀ cycloalkyl, C₁-C₂₀ heterocycloalkyl, aryl, or heteroaryl.

12. The compound of claim 1, wherein R₃ is halo or C₁-C₁₀ alkyl.

13. A method for treating a cannabinoid-receptor mediated disorder, comprising administering to a subject in need thereof an effective amount of a compound of formula (I): 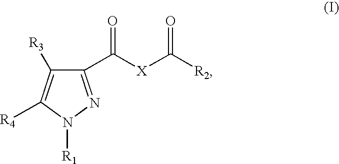
wherein
X is C(RₐR_{b}) or N(Rₐ), in which each of Rₐ and R_{b}, independently, is H, C₁-C₁₀ alkyl, C₃-C₂₀ cycloalkyl, C₁-C₂₀ heterocycloalkyl, aryl, or heteroaryl;
R₂ is H, halo, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, or NR_{c}R_{d}, in which each of R_{c} and R_{d}, independently, is H, C₁-C₁₀ alkyl, C₃-C₂₀ cycloalkyl, C₁-C₂₀ heterocycloalkyl, aryl, or heteroaryl; and
each of R₁, R₃, and R₄, independently, is H, halo, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl.

14. The method of claim 13, wherein X is CH₂ or NH.

15. The method of claim 14, wherein R₂ is C₁-C₁₀ alkyl, C₃-C₂₀ cycloalkyl, C₁-C₂₀ heterocycloalkyl, aryl, or NR_{c}R_{d}, in which each of R_{c} and R_{d}, independently, is H, C₁-C₁₀ alkyl, C₃-C₂₀ cycloalkyl, C₁-C₂₀ heterocycloalkyl, aryl, or heteroaryl.

16. The method of claim 15, wherein R₁ is aryl substituted with halo.

17. The method of claim 16, wherein R₁ is 2,4-dichlorophenyl.

18. The method of claim 13, wherein R₁ is 2,4-dichlorophenyl.

19. The method of claim 13, wherein R₄ is aryl or heteroaryl.

20. The method of claim 13, wherein R₂ is wherein R₂ is C₁-C₁₀ alkyl, C₃-C₂₀ cycloalkyl, C₁-C₂₀ heterocycloalkyl, aryl, or NR_{c}R_{d}, in which each of R_{c}, and R_{d}, independently, is H, C₁-C₁₀ alkyl, C₃-C₂₀ cycloalkyl, C₁-C₂₀ heterocycloalkyl, aryl, or heteroaryl.

21. The method of claim 13, wherein R₃ is halo or C₁-C₁₀ alkyl.

22. The method of claim 13, wherein the cannabinoid-receptor mediated disorder is liver fibrosis, obesity, metabolic syndrome, hyperlipidemia, type II diabetes, atherosclerosis, substance addiction, depression, motivational deficiency syndrome, learning or memory dysfunction, analgesia, haemorrhagic shock, ischemia, liver cirrhosis, neuropathic pain, antiemesis, high intraocular pressure, bronchodilation, osteoporosis, cancer, a neurodegenerative disease, or an inflammatory disease.

23. The method of claim 22, wherein the cannabinoid-receptor mediated disorder is obesity, metabolic syndrome, substance addiction, neuropathic pain, or an inflammatory disease.

24. The method of claim 22, wherein the cannabinoid-receptor mediated disorder is cancer.

25. The method of claim 24, wherein the cancer is prostate cancer, lung cancer, breast cancer, or head and neck cancer.
